(19) United States
(12) Patent Application Publication
(10) Pub. No.: US 2023/0276832 A1
(43) Pub. Date: Sep. 7, 2023

(54) ULTRASONIC PREPARATION METHOD OF PROTEIN-DERIVED PEPTIDE-POLYSACCHARIDE NANOPARTICLES LOADED WITH BIOACTIVE COMPONENTS

(71) Applicant: JIANGSU UNIVERSITY, Zhenjiang (CN)

(72) Inventors: Haile MA, Zhenjiang (CN); Xiaofeng REN, Zhenjiang (CN); Qiufang LIANG, Zhenjiang (CN); Xinru SUN, Zhenjiang (CN); Xueqiong LIU, Zhenjiang (CN); Jialin TANG, Zhenjiang (CN); Chengwei ZHOU, Zhenjiang (CN)

(73) Assignee: JIANGSU UNIVERSITY, Zhenjiang (CN)

(21) Appl. No.: 18/008,191

(22) PCT Filed: Jan. 11, 2022
(86) PCT No.: PCT/CN2022/071246

§ 371 Date: Dec. 5, 2022

(30) Foreign Application Priority Data
Apr. 27, 2021 (CN) 202110457751.6

(51) Int. Cl. A23L 5/30; A23L 33/18; A23L 33/105; A23L 29/275

(52) U.S. Cl. CPC A23L 5/32; A23L 33/18; A23L 33/105; A23L 29/275; A23V 2002/00

(57) ABSTRACT

An ultrasonic preparation method of protein peptide-polysaccharide nanoparticles loaded with bioactive components is conducted as follows: Chitosan solution containing 1% glacial acetic acid is added gently to the casein phosphopeptide solution containing quercetin stock solution in an equal volume under constant magnetic stirring; pH is adjusted accordingly before subjecting the mixture solution to ultrasonic treatment; after ultrasonication, the quercetin-loaded casein phosphopeptide-chitosan nanoparticles are obtained by freeze-drying. In the process of using the electrostatic interaction between the casein phosphopeptide and the chitosan to embed the quercetin, the present disclosure employed the multi-mode ultrasonic processing technology to facilitate the cross-linking of the polypeptide and the polysaccharide through the physical force of ultrasound. This enabled the complex encapsulation to incorporate additional bioactive components. The quercetin product exhibited excellent encapsulation efficiency, good water solubility, good light and thermal stability, and strong antioxidant properties, which significantly expanded the bioavailability of quercetin in the gastrointestinal tract.

### CROSS REFERENCE TO THE RELATED APPLICATIONS

This application is the national phase entry of International Application No. PCT/CN2022/071246, filed on Jan. 11, 2022, which is based upon and claims priority to Chinese Patent Application No. 202110457751.6, filed on Apr. 27, 2021, the entire contents of which are incorporated herein by reference.

### TECHNICAL FIELD

The present disclosure relates to the technical field of food encapsulation, and more specifically to a method for preparing composite nanoparticles by using casein phosphopeptide and chitosan as raw materials to load bioactive substance quercetin and employ ultrasonic treatment technology.

### BACKGROUND

Quercetin is a kind of flavonoid, which are a bioactive constituent. It possesses antioxidant and anti-tumor properties, as well as the ability to reduce blood pressure and blood lipids. However, quercetin's low water solubility, poor stability, and low bioavailability seriously restrict its application in the food and pharmaceutical industries. The construction of active compounds through nano-encapsulation to generate composite nanoparticles has been found in the literature to not only provide the human body with essential nutrients but also improve the water solubility, stability and bioavailability of hydrophobic substances.

Natural biological macromolecules such as proteins, polypeptides and polysaccharides are essential nutrients for the human body and have the advantages of high nutritional value, high safety, cheap cost, and easy accessibility. Numerous studies have indicated that they can serve as excellent carriers for active ingredients. According to research findings, the interaction between proteins and polysaccharides to form complexes can overcome the pH sensitivity, poor stability and low embedding efficiency of single components, and plays a significant role in embedding and protecting biologically active components. Therefore, domestic and international researchers have conducted extensive studies on the synthesis methods of protein-polysaccharide complexes and their encapsulation of bioactive components. Due to the folded condition of the natural protein and polysaccharide structures, the reactive groups remain encased within the macromolecules. The binding force of protein and polysaccharide appears modest, and nanoparticles generated by complex coagulation from them have poor functionalities. After enzymatic hydrolysis of proteins, polypeptides with a large number of strong reactive groups are obtained, which could readily form stable nano-scale colloidal complexes through physical interactions with active substance molecules, including hydrophobic interactions, hydrogen bonds and van der Waals forces. In terms of enhancing the bioavailability and stability of bioactive substances, the aforementioned complexes proved to be more favorable. The casein phosphopeptide obtained by enzymatic hydrolysis is a bioactive phosphoserine-rich peptide with the core structure -Ser(P)-Ser(P)-Ser(P)-Glu-Glu. Because it contained phosphoserine residues clustered collectively [-Ser(P)-], it might react with natural flavonoids. Therefore, casein phosphopeptides have a significant capacity to load flavonoids, which can overcome the problem of existing delivery systems' poor loading performance. Chitosan, the only positively charged polysaccharide, is a deacetylated derivative of chitin-containing amino groups. It has been widely used in many industries, such as food, medicine and cosmetics, because of its good hydrophilicity, biocompatibility and biodegradability, etc. In order to promote the efficient application of quercetin in food, medicine and other fields, researchers have constructed different delivery vehicles to improve the water solubility, processing properties and bioavailability of quercetin. Chen et al. {Chen H, Yao Y. Phytoglycogen improves the water solubility and Caco-2 monolayer permeation of quercetin [J]. Food Chemistry, 2017, 221(April 15):248-257.} encapsulated quercetin using phytoglycogen and constructed a nano-delivery strategy to increase its solubility. However, in this process, 25% ethanol is added, which increased the preparation cost and is not conducive to subsequent freeze-dried. Zhang et al. {Zhang Y, Yang Y, Tang K, et al. Physicochemical characterization and antioxidant activity of quercetin-loaded chitosan nanoparticles[J]. Journal of Applied Polymer Science, 2010, 107(2):891-897.} synthesized quercetin-loaded nanoparticles by ion gelation of chitosan and tripolyphosphate, although their antioxidant activity is low, with a DPPH free radical scavenging rate of approximately 50%. Yan et al. {Yan L, Wang R, Wang H, et al. Formulation and characterization of chitosan hydrochloride and carboxymethyl chitosan encapsulated quercetin nanoparticles for controlled applications in foods system and simulated gastrointestinal condition[J]. Food Hydrocolloids, 2018, 84(November): 450-457.} developed quercetin-loaded chitosan hydrochloride (CHC) and carboxymethyl chitosan (CMCN) nanoparticles using electrostatic interaction; nevertheless, the obtained nanoparticles had a larger average size of about 386.30±10.10 nm.

In the present disclosure, negatively charged casein phosphopeptide and positively charged chitosan are combined to produce nanoparticles with a stable structure for encapsulating quercetin, and advanced multi-mode ultrasonic processing technology is employed. It is anticipated that ultrasound could stimulate the two biological macromolecules; polypeptide and polysaccharide to generate resonance frequencies that correspond with their inherent frequencies. Hence, cross-linking occurs, leading to substantial encapsulation efficiency, good water solubility, small average particle size (about 241.27±7.63 nm), good photothermal stability, strong oxidation resistance and high bioavailability.

### SUMMARY

In order to address the aforementioned issues, the present disclosure prepared quercetin-loaded casein phosphopeptide-chitosan composite nanoparticles by developing a casein phosphopeptide-chitosan loading system utilizing a physical processing method of ultrasonic treatment. Furthermore, the impact of sonication conditions on the encapsulation of quercetin and the properties of its composite nanoparticles are investigated.

The ultrasonic preparation method for the present disclosure's quercetin-loaded casein phosphopeptide-chitosan composite nanoparticles consisted of the following steps:
(1) The casein phosphopeptide is dissolved in distilled water, and the pH of the solution is adjusted to 11;
(2) Chitosan is dissolved in 1% glacial acetic acid and agitated magnetically until completely dissolved;
(3) Quercetin stock solution is prepared by dissolving quercetin into absolute ethanol;
(4) In accordance with various mass ratios of quercetin and casein phosphopeptide, the casein phosphopeptide solution from step (1) is slowly added to the quercetin stock solution from step (3) under constant speed stirring at room temperature;
(5) Depending on the mass ratios of casein phosphopeptide and chitosan, an equal volume of the chitosan solution from step (2) is added dropwise to the mixture from step (4) maintaining the same stirring condition; the pH is adjusted to 6;
(6) In step (5), the combined solution is subjected to ultrasonic treatment; after completion of ultrasonication, a quercetin-loaded casein phosphopeptide-chitosan nanoparticle dispersion is obtained; and finally freeze-drying yielded a quercetin-loaded casein phosphopeptide-chitosan nanoparticle. Wherein the concentration of casein phosphopeptide described in step (1) is 1.0-3.0 mg/mL, with a preferred value of 1.5 mg/mL.

Whereby a mass ratio of quercetin and casein phosphopeptide specified in step (4) is 1:5-1:20, and most preferably 1:15.

In which the mass ratio of casein phosphopeptide and chitosan stated in step (5) is (1-3):(1-3), ideally 1:1.

However, the specific parameters of the ultrasonic treatment in step (6) are 20 kHz, 35 kHz, 50 kHz, 20/35 kHz, 20/50 kHz, 35/50 kHz, and 20/35/50 kHz, in which, ultrasonic frequency of 35/50 kHz is selected. Likewise, other selected parameters are ultrasonic power with a range of 60 W-300 W and treatment time (5-30 min), while 240 W and 15 min are optimized, respectively; the ultrasonic intermittent ratio is 30 s/5 s.

The beneficial effects of the present disclosure are as follows:
(1) In order to develop a polypeptide-polysaccharide loading system, chitosan is added to the casein phosphopeptide in the present disclosure. The polypeptide and the polysaccharide are cross-linked to form small aggregates and electrostatic interaction played a vital role in their cohesion. Each aggregate further formed composite particles through hydrophobic interactions, which provided a basis for the encapsulation of bioactive components.
(2) Ultrasonic-induced casein phosphopeptide-chitosan composite loading of quercetin nanoparticles could significantly improve the water solubility of quercetin, with the maximum value increasing by approximately 39.76 μg/mL. The above-prepared nanoparticles had the advantages of high entrapment rate, good stability, potent anti-oxidation, extended sustained release time, and high bioavailability. It could be used in many industries, including food, health care products, medicines and cosmetics.
(3) In the present disclosure, the ultrasonic treatment method is used in the process of encapsulating quercetin with a casein phosphopeptide-chitosan mixture. Ultrasonic treatment is a sustainable and environmentally favorable processing approach that has been extensively used in the food industry. Ultrasound is a novel physical treatment method for the preparation of nano-delivery systems. The physical force of ultrasound promotes the mutual cross-linking of polypeptides and polysaccharides, hence improving the embedding effect of bioactive components.
(4) In accordance with the present disclosure, the ultrasonic preparation method for the quercetin-loaded casein phosphopeptide-chitosan composite nanoparticles is straightforward and suitable for industrial production. And casein phosphopeptide and chitosan are inexpensive raw materials.

### BRIEF DESCRIPTION OF THE DRAWINGS

FIG. 1 depicts the structure of the multi-mode ultrasonic biological treatment equipment of the present disclosure, wherein 1, 2, and 3 are ultrasonic vibration plates, 4 is a water container, 5 is a water bath, 6 is a temperature probe, 7 is a circulating pump, and 8 is a computer program controller, 9, 10, 11 are ultrasonic controllers.

FIGS. 2A-2J are scanning electron microscope images of the nanoparticles (×20000 times) of raw materials prepared under different conditions. FIG. 2A, FIG. 2B, and FIG. 2C represent the original powders of Qu, CS, and CPP, respectively. FIG. 2D, FIG. 2E, FIG. 2F, FIG. 2G, FIG. 2H, FIG. 2I, and FIG. 2J are each composed of a single casein phosphopeptide nanoparticles (CPP), quercetin-loaded casein phosphopeptide nanoparticles (CPP-Qu(15:1)), casein phosphopeptide-chitosan nanoparticles (CPP-CS(1:1)), quercetin-loaded casein phosphopeptide-chitosan nanoparticles (CPP-CS-Qu(15:15:1)), quercetin-loaded casein phosphopeptide-chitosan nanoparticles prepared by ultrasound treatment (CPP-CS-Qu(US) (15:15:1)), quercetin-loaded casein phosphopeptide-chitosan nanoparticles (CPP-CS-Qu(5:5:1)), sonicated quercetin-loaded casein phosphopeptide-chitosan nanoparticles (CPP-CS-Qu(US)(5:5:1)), respectively.

FIG. 3 is the Fourier transform infrared spectrum of the nanoparticle of raw materials and the nanoparticle prepared under different conditions. The spectra from bottom to top represented single casein phosphopeptide nanoparticles (CPP), CS and Qu raw powder, casein phosphopeptide-chitosan nanoparticles (CPP-CS(1:1)), loaded quercetin casein phosphopeptide nanoparticles (CPP-Qu(15:1)), quercetin-loaded casein phosphopeptide-chitosan nanoparticles (CPP-CS-Qu(15:15:1)), sonication quercetin-loaded casein phosphopeptide-chitosan nanoparticles (CPP-CS-Qu(US) (15:15:1)), quercetin-loaded casein phosphopeptide-chitosan nanoparticles (CPP-CS-Qu(5:5:1)), sonicated quercetin-loaded casein phosphopeptide-chitosan nanoparticles (CPP-CS-Qu(US)(5:5:1)) spectrum.

FIG. 4 showed the X-ray diffraction patterns of nanoparticles with raw materials and the nanoparticle prepared under different conditions. The spectra from bottom to top represented single casein phosphopeptide nanoparticles (CPP), CS and Qu raw powder, casein phosphopeptide-chitosan nanoparticles (CPP-CS(1:1)), loaded quercetin casein phosphopeptide nanoparticles (CPP-Qu(15:1)), quercetin-loaded casein phosphopeptide-chitosan nanoparticles (CPP-CS-Qu(15:15:1)), sonication quercetin-loaded casein phosphopeptide-chitosan nanoparticles (CPP-CS-Qu(US) (15:15:1)), quercetin-loaded casein phosphopeptide-chitosan nanoparticles (CPP-CS-Qu(5:5:1)), sonicated quercetin-loaded casein phosphopeptide-chitosan nanoparticles (CPP-CS-Qu(US)(5:5:1)) spectrum.

FIG. 5 showed the effect of different preparation conditions on the water solubility of quercetin. The mass ratio of quercetin to casein phosphopeptide is 1:0, representing free quercetin, and the rest is casein phosphopeptide loaded with quercetin-chitosan nanoparticles (US means sonication).

FIG. 6 illustrated the impact of temperature on the stability of free quercetin in quercetin and quercetin-loaded composite nanoparticles. The histograms from left to right represented free quercetin (Free-Qu), quercetin-loaded casein phosphopeptide nanoparticles (CPP-Qu), quercetin-loaded casein phosphopeptide-chitosan nanoparticles (CPP-CS-Qu), and sonicated quercetin-loaded casein phosphopeptide-chitosan nanoparticles (CPP-CS-Qu (US)), respectively.

FIG. 7 showed the effect of light on the stability of quercetin in free quercetin and quercetin-loaded composite nanoparticles. The line graphs from bottom to top represented free quercetin (Free-Qu), quercetin-loaded casein phosphopeptide nanoparticles (CPP-Qu), quercetin-loaded casein phosphopeptide-chitosan nanoparticles (CPP-CS-Qu), and sonicated quercetin-loaded casein phosphopeptide-chitosan nanoparticles (CPP-CS-Qu (US)), respectively.

FIG. 8 shows the scavenging ability of nanoparticles to DPPH radicals. The histograms from left to right represented casein phosphopeptide (CPP), casein phosphopeptide-chitosan nanoparticles (CPP-CS), quercetin free in ethanol (Qu-ethanol), and free in the water. Quercetin (Qu-water), quercetin-loaded casein phosphopeptide nanoparticles (CPP-Qu), quercetin-loaded casein phosphopeptide-chitosan nanoparticles (CPP-CS-Qu), sonicated quercetin-loaded casein phosphopeptide-chitosan nanoparticles (CPP-CS-Qu (US)).

FIG. 9 depicts the ability of nanoparticles to scavenge ABTS free radicals. Left to right, the histograms represented casein phosphopeptide (CPP), casein phosphopeptide-chitosan nanoparticles (CPP-CS), quercetin free in ethanol (Qu-ethanol), and free in the water. Quercetin (Qu-water), quercetin-loaded casein phosphopeptide nanoparticles (CPP-Qu), quercetin-loaded casein phosphopeptide-chitosan nanoparticles (CPP-CS-Qu), sonicated quercetin-loaded casein phosphopeptide-chitosan nanoparticles (CPP-CS-Qu (US)).

FIG. 10 exhibits the release profile of quercetin during in vitro gastrointestinal digestion simulation. The line graphs from top to bottom represented free quercetin (Free-Qu), quercetin-loaded casein phosphopeptide nanoparticles (CPP-Qu), quercetin-loaded casein phosphopeptide-chitosan nanoparticles (CPP-CS-Qu), and sonicated quercetin-loaded casein phosphopeptide-chitosan nanoparticles (CPP-CS-Qu (US)), respectively.

### DETAILED DESCRIPTION OF THE EMBODIMENTS

Unless otherwise specified, terms used in the present disclosure are generally understood by individuals with ordinary skills in the art. The present disclosure will be described in further detail below in association with specific embodiments and data. It is emphasized that the purpose of these examples is only to illustrate the present disclosure and not to limit its scope in any manner. It is emphasized that the purpose of these examples is just to explain the present disclosure and not to limit its scope in any manner.

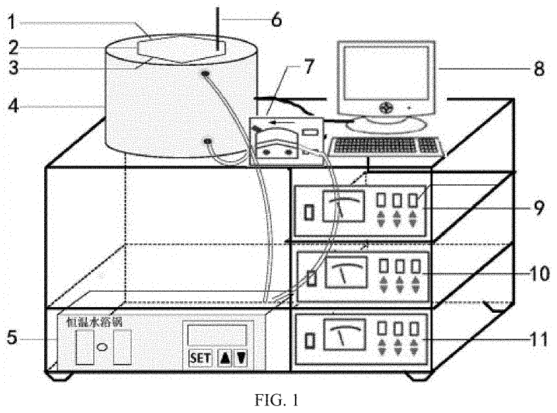

FIG. 1 is a schematic diagram of the multi-mode ultrasonic biological treatment equipment of the present disclosure. The device was equipped with a computer program controller 8. The ultrasonic working parameters, such as ultrasonic power density, frequency, pulse working time, intermittent time and total processing time can be configured to control three ultrasonic controllers 9, 10, and 11 respectively. Which connected three ultrasonic vibration plates 1, 2, and 3 with different frequencies to accomplish single frequency/double frequency/triple frequency ultrasonic treatment. The solution to be treated was placed in liquid container 4 for single-frequency/dual-frequency/multi-frequency ultrasonic treatment, and the circulating pump 7 was activated to circulate the solution. The temperature of the solution was automatically controlled by the water bath 5 and the temperature probe 6.

Experimental Materials:

Casein phosphopeptides were purchased from Shanghai Yien Chemical Technology Co., Ltd. Analytical grades such as chitosan and quercetin were purchased from Sinopharm Chemical Reagent Co., Ltd.

The following procedures were used to evaluate the encapsulation efficiency and the loading rate of the quercetin-loaded composite nanoparticles prepared according to the examples of the present disclosure: the prepared quercetin-loaded composite nanoparticles dispersion was centrifuged at 10,000 rpm at 4° C. for 20 min. After discarding insoluble quercetin and large aggregates, a portion of the supernatant and an appropriate multiple of absolute ethanol were taken, respectively. The resulting mixture was extracted by vortexing for 5 min and then centrifuged at 4° C. and 10,000 rpm for 5 min. The absorbance of the supernatant was measured at 374 nm. The encapsulated quercetin content was calculated according to the standard curve and the dilution factor. The measured absorbance value of the sample without quercetin was used as a blank control. The encapsulation efficiency and loading rate of quercetin were calculated as follows:

$$Encapsulation efficiency ( \% ) = \frac{\begin{matrix}Encapsulated Quercetin \\ Content\end{matrix}}{\begin{matrix}Total content of \\ added quercetin\end{matrix}} \times 100 \% \\ Loading rate ( \% ) = \frac{Encapsulated Quercetin Content}{Total weight of the sample} \times 100 \%$$

### Example 1: Optimization of Casein Phosphopeptide Concentration for the Synthesis of Quercetin-Loaded Casein Phosphopeptide-Chitosan Composite Nanoparticles

(1) Casein phosphopeptide was dissolved in distilled water at a concentration of 1.0, 1.5, 2.0, 2.5, 3.0 mg/mL, and the pH of the solution was adjusted to 11;
(2) Chitosan was dissolved in 1% glacial acetic acid solution, and magnetically stirred until completely dissolved;
(3) Quercetin was dissolved in absolute ethanol to prepare a queretin stock solution;
(4) The casein phosphopeptide solution in step (1) was slowly added to the quercetin stock solution in step (3) according to the mass ratio of quercetin and casein phosphopeptide being 1:15 under constant stirring;
(5) Under the condition of stirring at room temperature, according to the mass ratio of casein phosphopeptide and chitosan of 1:1, an equal volume of step (2) chitosan solution was added dropwise to the mixture of step (4); its pH was adjusted to 6;
(6) The mixed solution in step (5) was subjected to ultrasonic treatment. After ultrasound treatment, a quercetin-loaded casein phosphopeptide-chitosan nanoparticle dispersion was obtained.

The concentration of casein phosphopeptide was optimized according to the encapsulation efficiency and loading rate. The results were shown in Table 1. The encapsulation efficiency and loading rate of quercetin increased and then declined with increasing concentrations of casein phosphopeptide. When the concentration of casein phosphopeptide was 1.5 mg/mL, quercetin had the maximum encapsulation effect, the encapsulation efficiency of 65.90%, and a loading rate of 2.20%. Using the encapsulation efficiency and quercetin loading rate as the main indicators, the concentration of casein phosphopeptide was determined to be 1.5 mg/mL, and the preparation procedure was then optimized.

TABLE 1
Effect of casein phosphopeptide concentration on quercetin
encapsulation efficiency and loading rate

| | Casein phosphopeptide concentration (mg/mL) | | | | |
|---|---|---|---|---|---|
| | 1.0 | 1.5 | 2.0 | 2.5 | 3.0 |
| Encapsulation | 59.81 | 65.90 | 61.12 | 56.80 | 53.84 |
| efficiency (%) | | | | | |
| Loading rate (%) | 1.99 | 2.20 | 2.04 | 1.89 | 1.79 |

### Example 2: Optimization of the Casein Phosphopeptide Mass Ratio to Chitosan in the Synthesis of Quercetin-Loaded Casein Phosphopeptide-Chitosan Composite Nanoparticles

(1) The concentration of casein phosphopeptide dissolved in distilled water was 1.5 mg/mL, and the adjusted pH was 11;
(2) Chitosan was dissolved in 1% glacial acetic acid solution, and stirred magnetically at room temperature until completely dissolved;
(3) Quercetin was dissolved in absolute ethanol to prepare a quercetin stock solution;
(4) The casein phosphopeptide solution in step (1) was slowly added to the quercetin stock solution in step (3) according to the mass ratio of quercetin and casein phosphopeptide being 1:15 under constant stirring;
(5) Under the same stirring condition, in accordance with the mass ratio of casein phosphopeptide and chitosan (it could be 1:0, 1:3, 1:2, 1:1, 2:1, 3:1), an equal volume of step (2) chitosan solution was added dropwise to the mixture of step (4); its pH was adjusted to 6;
(6) The mixed solution in step (5) was subjected to ultrasonic treatment. After ultrasound treatment, a quercetin-loaded casein phosphopeptide-chitosan nanoparticle dispersion was obtained.

The optimization of the mass ratio of casein phosphopeptide to chitosan was shown in Table 2. Compared to the control (without chitosan), the inclusion of chitosan significantly improved the encapsulation efficiency and loading rate of quercetin by casein phosphopeptide. Based on the principle of encapsulation efficiency, when the mass ratio of casein phosphopeptide to chitosan was 1:1, the quercetin encapsulation efficiency reached a maximum of 65.48%; while the loading rate was 2.18%. In summary, the mass ratio of casein phosphopeptide to chitosan was selected to be 1:1 in order to optimize the subsequent production procedure.

TABLE 2
Effect of mass ratio of casein phosphopeptide and chitosan
on quercetin encapsulation efficiency and loading rate

| | Mass ratio of casein phosphopeptide to chitosan | | | | | |
|---|---|---|---|---|---|---|
| | 1:0 | | | | | |
| | (control) | 1:3 | 1:2 | 1:1 | 2:1 | 3:1 |
| Encapsulation | 46.55 | 58.59 | 63.08 | 65.48 | 62.14 | 56.32 |
| efficiency (%) | | | | | | |
| Loading rate (%) | 3.10 | 0.98 | 1.40 | 2.18 | 2.76 | 2.82 |

### Example 3: Optimization of the Mass Ratio of Quercetin to Casein Phosphopeptide in the Preparation of Quercetin-Loaded Casein Phosphopeptide-Chitosan Composite Nanoparticles

(2) At pH 11, casein phosphopeptide was dissolved in distilled water at a concentration of 1.5 mg/mL.
(2) Chitosan was dissolved in 1% glacial acetic acid solution, and magnetically stirred to dissolve properly;
(3) Quercetin was dissolved in absolute ethanol to prepare a quercetin stock solution;
(4) According to the fixed mass ratio of quercetin to casein phosphopeptide (1:5, 1:10, 1:12.5, 1:15, 1:20), the casein phosphopeptide solution of step (1) was slowly added to the quercetin stock solution in step (3) under constant speed stirring;
(5) In accordance with the mass ratio of casein phosphopeptide and chitosan 1:1, an equivalent volume of step (2) chitosan solution was added dropwise to the mixture of step (4) under the same stirring condition, and its pH was adjusted to 6;
(6) The mixed solution in step (5) was treated with ultrasonic treatment. After ultrasound treatment, a quercetin-loaded casein phosphopeptide-chitosan nanoparticle dispersion was obtained.

The optimized mass ratio of quercetin to casein phosphopeptide was shown in Table 3. As the mass ratio of quercetin to casein phosphopeptide decreased from 1:10 to 1:20, the encapsulation efficiency of quercetin increased first and then decreased, while the loading rate also decreased. When the mass ratio was 1:15, the encapsulation rate of quercetin reached a maximum of 65.72%, and the corresponding loading rate was 2.50%. Considering the quercetin encapsulation efficiency as the main indicator, the mass ratio of quercetin and casein phosphopeptide was selected as 1:15 to optimize the next preparation process.

TABLE 3
Effects of quercetin and casein phosphopeptide mass ratio
on quercetin encapsulation efficiency and loading rate

| | Mass ratio of casein phosphopeptide to chitosan | | | | |
|---|---|---|---|---|---|
| | 1:5 | 1:10 | 1:12.5 | 1:15 | 1:20 |
| Encapsulation | 21.46 | 57.96 | 62.42 | 65.72 | 62.12 |
| efficiency (%) | | | | | |
| Loading rate (%) | 2.15 | 2.90 | 2.50 | 2.19 | 1.55 |

### Example 4: Ultrasonic Frequency Optimization to Prepare Quercetin-Loaded Casein Phosphopeptide-Chitosan Composite Nanoparticles

(1) Casein phosphopeptide was dissolved in distilled water in a concentration of 1.5 mg/mL, and the adjusted pH was 11;
(2) Chitosan was dissolved in 1% glacial acetic acid solution, and completely dissolved by using a magnetic stirrer;
(3) Quercetin was dissolved in absolute ethanol to prepare a quercetin stock solution;
(4) According to the fixed mass ratio of quercetin to casein phosphopeptide 1:15, the solution prepared in step (1) was slowly added to the solution in step (3) under constant speed stirring;
(5) Under the condition of stirring at room temperature, according to the mass ratio of 1:1 between casein phosphopeptide and chitosan, an equal volume of step (2) chitosan solution was added dropwise to the mixture of step (4), its pH was adjusted to 6;
(6) The mixed solution of step (5) was treated with ultrasound. The ultrasonic frequency was 20, 35, 50, 20/35, 20/50, 35/50, 20/35/50 kHz, the ultrasonic power was 180 W, the ultrasonic time was 10 min, and the ultrasonic interval was 30 s/5 s. The quercetin-loaded casein phosphopeptide-chitosan nanoparticle dispersion was obtained after ultrasonication.

The optimization of ultrasonic frequency was shown in Table 4. It could be seen that after the mixed solution was treated with different ultrasonic frequencies. The dual-frequency synchronous ultrasonic treatment with the ultrasonic frequency of 35/50 produced the highest quercetin encapsulation efficiency; its encapsulation efficiency and loading rates were 68.96% and 2.30%, respectively. Therefore, the ultrasonic frequency of 35/50 kHz was selected to optimize of the next preparation step.

TABLE 4
Effects of ultrasonic frequency on quercetin encapsulation efficiency and loading rate

| Ultrasonic frequency (kHz) | Control | 20 | 35 | 50 | 20/35 | 20/50 | 35/50 | 20/35/50 |
|---|---|---|---|---|---|---|---|---|
| Encapsulation efficiency | 64.33 | 66.65 | 65.44 | 65.36 | 65.78 | 65.64 | 68.96 | 66.17 |
| (%) | | | | | | | | |
| Loading rate (%) | 2.14 | 2.22 | 2.18 | 2.18 | 2.19 | 2.19 | 2.30 | 2.21 |

### Example 5: Optimization of Ultrasonic Power in the Synthesis of Quercetin-Loaded Casein Phosphopeptide-Chitosan Composite Nanoparticles

(1) Casein phosphopeptide was dissolved in distilled water, the concentration of casein phosphopeptide was 1.5 mg/mL, and the pH of the solution was adjusted to 11;
(2) Chitosan was dissolved in 1% glacial acetic acid solution, and magnetically stirred until completely dissolved;
(3) Absolute ethanol was used to prepare quercetin stock solution;
(4) According to the fixed mass ratio of quercetin to casein phosphopeptide 1:15, the casein phosphopeptide solution of step (1) was slowly added to the quercetin stock solution in step (3) under constant speed stirring;
(5) Under the condition of stirring at room temperature, according to the mass ratio of casein phosphopeptide and chitosan 1:1, an equal volume of step (2) chitosan solution was added dropwise to the mixture of step (4); its pH was adjusted to 6;
(6) The mixed solution of step (5) was treated with ultrasound. The ultrasonic frequency was 35/50 kHz, the ultrasonic power was 160, 120, 180, 240, and 300 W, the ultrasonic time was 10 min, and the ultrasonic interval was 30 s/5 s. The quercetin-loaded casein phosphopeptide-chitosan nanoparticle dispersion was obtained after ultrasonication.

The optimization of ultrasonic power can be seen in Table 5. With the increase of ultrasonic power, the encapsulation efficiency and loading rate of quercetin showed a trend of increasing first and then decreasing. At 240 W ultrasonic power maximum encapsulation efficiency was 72.25%, and the load rate was 2.41%. The ultrasonic power of 240 W was selected to optimize the next preparation process.

TABLE 5
Effects of ultrasonic power on quercetin
encapsulation efficiency and loading rate

| | Ultrasonic power (W) | | | | | |
|---|---|---|---|---|---|---|
| | Control | 60 | 120 | 180 | 240 | 300 |
| Encapsulation | 65.03 | 66.70 | 67.81 | 68.37 | 72.25 | 62.29 |
| efficiency (%) | | | | | | |
| Loading rate (%) | 2.17 | 2.22 | 2.26 | 2.28 | 2.41 | 2.08 |

### Example 6: Optimization of Ultrasonic Time to Prepare Quercetin-Loaded Casein Phosphopeptide-Chitosan Composite Nanoparticles

(1) Casein phosphopeptide was dissolved in distilled water, where casein phosphopeptide concentration was 1.5 mg/mL, and the solution pH was maintained at 11;
(2) Chitosan was dissolved in glacial acetic acid solution (1%), and magnetically stirred until completely dissolved;
(3) Quercetin was dissolved in absolute ethanol to prepare a quercetin stock solution;
(4) According to the fixed mass ratio of quercetin to casein phosphopeptide 1:15, the casein phosphopeptide solution of step (1) was slowly added to the quercetin stock solution in step (3) under constant speed stirring;
(5) According to the mass ratio of casein phosphopeptide and chitosan 1:1, an equal volume of step (2) chitosan solution was added dropwise to the mixture of step (4) in a constant stirring at room temperature; the adjusted pH was 6;
(6) Ultrasonic treatment was given to the mixture of the solution from step (5). The ultrasonic frequency was 35/50 kHz; the ultrasonic power was 240 W; the ultrasonic time was 5, 10, 15, 20, and 30 min; and the ultrasonic interval was 30 s/5 s. After the ultrasonication, the quercetin-loaded casein phosphopeptide-chitosan nanoparticle dispersion was obtained.

Ultrasonic time optimization was depicted in Table 6. Initially, with the extension of the ultrasonic time, the quercetin encapsulation efficiency and the loading rate exhibited an increasing trend; however, after a certain period of time, the trend reversed. When the ultrasonic time was 15 min, the quercetin encapsulation efficiency and loading rate simultaneously reached their maximum value. Therefore, 15 min of ultrasonic time was selected to optimize the subsequent preparation process.

TABLE 6
Effects of ultrasonic time on quercetin
encapsulation efficiency and loading rate

| | Ultrasound time (min) | | | | | |
|---|---|---|---|---|---|---|
| | Control | 5 | 10 | 15 | 20 | 30 |
| Encapsulation | 64.35 | 65.50 | 70.68 | 73.19 | 66.69 | 66.37 |
| efficiency (%) | | | | | | |
| Loading rate (%) | 2.14 | 2.18 | 2.36 | 2.44 | 2.22 | 2.21 |

### Experimental Example 1: Structural Characterization and Analysis of Quercetin-Loaded Composite Nanoparticles

In experimental examples 1 and 2, the ultrasonic preparation method for the quercetin-loaded casein phosphopeptide-chitosan composite nanoparticles was carried out as follows:
(1) Casein phosphopeptide was dissolved in distilled water (1.5 mg/mL) and the pH of the solution was adjusted to 11;
(2) Chitosan was dissolved in 1% glacial acetic acid solution, and completely dissolved by magnetic stirrer;
(3) Quercetin stock solution was prepared by dissolving quercetin in absolute ethanol;
(4) At a fixed mass ratio (1:15) of quercetin to casein phosphopeptide, the casein phosphopeptide solution from step (1) was slowly added to the quercetin stock solution in step (3) under constant speed stirring;
(5) Under the condition of stirring at room temperature, according to the mass ratio of casein phosphopeptide and chitosan 1:1, an equal volume of step (2) chitosan solution was added dropwise to the mixture of step (4); at pH6;
(6) The mixture solution of step (5) was treated ultrasonically. When the mass ratio of quercetin to casein phosphopeptide was 1:15, the preferred ultrasonic parameters were frequency 20/35/50 kHz, power 240 W, time 20 min, and intermittent ratio 20 s/5 s. When the mass ratio of quercetin to casein phosphopeptide was 1:5, the optimal ultrasonic conditions were ultrasonic frequency 20/35/50 kHz, power 300 W, time 15 min, and intermittent ratio 30 s/5 s. After ultrasound treatment, the quercetin-loaded casein phosphopeptide-chitosan nanoparticle dispersion was obtained, including (CPP-CS-Qu(US) (15:15:1) and CPP-CS-Qu(US) (5:5:1)). The nanoparticles prepared with a mass ratio of quercetin and casein phosphopeptide of 1:15 were named CPP-CS-Qu(US) (15:15:1); the nanoparticles prepared with a mass ratio of quercetin to casein phosphopeptide of 1:5 was named CPP-CS-Qu(US) (5:5:1).

Note: When using the above method to prepare the free quercetin (Free-Qu) dispersion, casein phosphopeptide nanoparticles (CPP), casein phosphopeptide-chitosan nanoparticles (CPP-CS (1:1)) and quercetin-loaded casein phosphopeptide nanoparticles (CPP-Qu (15:1)) dispersion, deionized water was used to replace the excluded biomacromolecule solution, and it was used as a control.

(1) Scanning Electron Microscope Analysis

Experimental conditions: An appropriate amount of freeze-dried samples were uniformly dispersed and fixed on the sample holder using double-sided carbon tape, and coated with a thin layer of gold. The microscopic morphology was observed at a magnification of 20,000 times at an accelerating voltage of 5 kV.

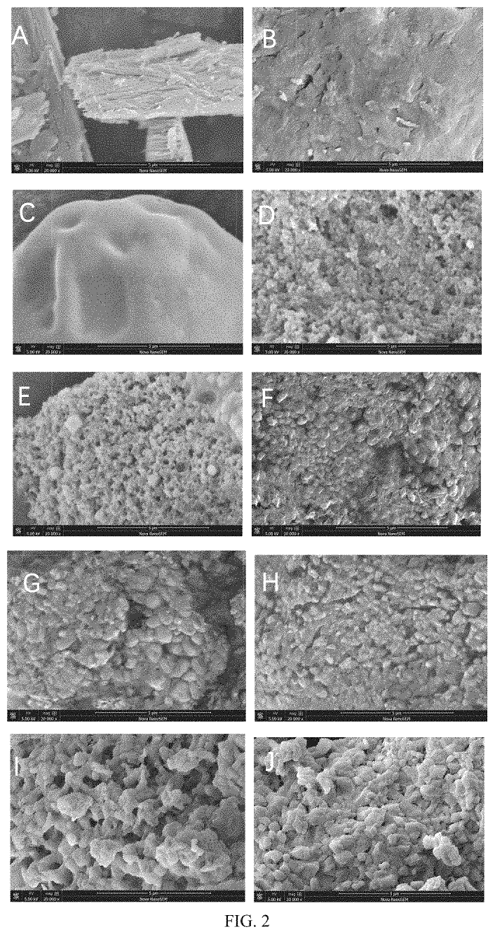

The microscopic morphology and particle size of the nanoparticles were observed intuitively by scanning electron microscope. FIGS. 2A-2J revealed that the surface of the quercetin monomer was rough and had an aggregated crystal surface. The surface of chitosan showed a smooth membrane structure, but the powder particles of casein phosphopeptide had irregular pits on the surface. The single CPP nanoparticles after lyophilization were typical regular spherical shapes with uniform particle distribution, and the particle size was about 150 nm. After the addition of quercetin to casein phosphopeptides, the size and morphology of CPP-Qu (15:1) particles and CPP nanoparticles were not significantly different. However, irregular, tightly packed aggregates were detected, which might have resulted from the interaction of casein phosphopeptide with quercetin. The ability of a single casein phosphopeptide to encapsulate quercetin was restricted, and excess quercetin might interfere with the intermolecular entanglement between polypeptide chains, preventing them from aggregating into spheres after solvent evaporation.

FIG. 2G & FIG. 2I were CPP-CS-Qu (15:15:1) and CPP-CS-Qu (5:5:1) nanoparticles, respectively. The morphology of CPP-CS-Qu system particles did not differ considerably from those of the CPP-CS system when quercetin was encapsulated by casein phosphopeptide and chitosan. Comparing CPP-CS-Qu (5:5:1) particles with CPP-CS-Qu (15:15:1) particles, the increase in quercetin content resulted in more severe agglomeration and an increase in the average particle size. (H) and (J) were the respective figure of CPP-CS-Qu(US) (15:15:1) and CPP-CS-Qu(US) (5:5:1) nanoparticles, prepared by sonication. Compared with the control, the surfaces of the nanoparticles prepared by ultrasonic treatment were smoother. The aggregation between particles was greatly reduced; the average particle size of the particles was significantly reduced, and the size distribution was more uniform. It could be seen from FIGS. 2A-2J that the average particle size of CPP-CS-Qu(US) (15:15:1) was around 250 nm. It can be illustrated that the shear force formed by microfluidics and shock waves generated by ultrasonic cavitation may alter the spatial structure of the casein phosphopeptide molecules, leading to a higher exposure of chemical bonds and an emergence in quercetin and casein phosphopeptides interaction sites. Thus, the resulting nanoparticles had a more compact and dense structure. Furthermore, effective ultrasonic treatment could promote the dispersion of the nanoparticles, thereby enhancing the monodispersity of the particles.

(2) Fourier Transform Infrared Spectroscopy Analysis

Experimental conditions: 1 mg of dry sample and 100 mg of KBr powder were fully ground in an agate mortar, mixed uniformly, pressed into a transparent sheet with a pellet machine and placed in an infrared spectrometer. The KBr pellet without a sample was used as the background blank. The scanning range was 800-4000 cm⁻¹; the resolution was 4 cm⁻¹, and the sample spectrum was obtained by scanning 36 times.

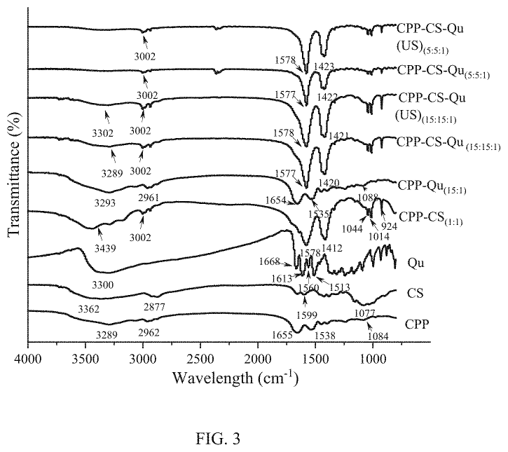

Fourier transform infrared spectroscopy has been used to study the interaction between casein phosphopeptide and chitosan and quercetin functional groups. As shown in FIG. 3, casein phosphopeptide, chitosan and quercetin showed specific broad peaks at 3289, 3362 and 3300 cm⁻¹, respectively, which were attributed to the stretching vibration of —OH. While at different mass ratios the casein phosphopeptide, chitosan and quercetin showed that the stretching vibration peaks of the hydroxyl group moved to 3439 cm⁻¹ (CPP-CS; 1:1), 3293 cm⁻¹ (CPP-Qu; 15:1) and 3289 cm⁻¹ (CPP-CS-Qu; 15:15:1), indicating the existence of hydrogen bonding between casein phosphopeptides and chitosan and quercetin. Hydrogen bonds in CPP-CS nanoparticles might be formed by the interaction between —OH on the chitosan molecular chain and —COO— and —PO3- groups on the casein phosphopeptide.

Compared with the single casein phosphopeptide, the peak at 1655 cm⁻¹ disappeared and the peak of amide II shifted from 1538 to 1578 cm⁻¹ in CPP-CS nanoparticles. While the peaks of amide I and amide II bands in CPP-Qu nanoparticles shifted from 1655 cm⁻¹ and 1538 cm⁻¹ to 1654 cm⁻¹ and 1535 cm⁻¹, respectively. The above results indicated that there was not only electrostatic interaction but also hydrophobic interaction between casein phosphopeptide and chitosan. The hydrophobic interaction between casein phosphopeptide and quercetin might be due to the interaction between the non-polar amino acids present in casein phosphopeptide and the aromatic ring of quercetin. Similarly, in comparison to single casein phosphopeptide and chitosan, the peaks of CPP-CS nanoparticles at 2962 cm-land 2877 cm⁻¹ have been red-shifted to 3002 cm⁻¹; and an obvious new infrared absorption peak has appeared at 1412 cm⁻¹. This peak should be a distinctive peak formed by cross-linking between —NH₃+ in the chitosan molecule with —COO— and —P═O— ion groups in the casein phosphopeptide molecule. This phenomenon also existed in the CPP-CS-Qu nanoparticle spectrum. In addition, FIG. 3 revealed that numerous sharp peaks of quercetin in the 800-1500 cm⁻¹ region disappeared in the CPP-CS-Qu nanoparticle spectrum, indicating that quercetin was successfully encapsulated in the CPP-CS nanocomposite.

After ultrasonication, the peak of the hydroxyl group in the infrared spectrum of CPP-CS-Qu (US) nanoparticles was significantly red-shifted from 3289 cm⁻¹ to 3302 cm⁻¹ (CPP-CS-Qu (US) (15:15:1)). Its amide peaks of band II shifted from 1577 cm⁻¹ to 1578 cm⁻¹ (CPP-CS-Qu (US) (15:15:1), CPP-CS-Qu (US) (5:5:1), respectively). And the new peaks at 1412 cm⁻¹ in CPP-CS was red-shifted from 1420 cm⁻¹ to 1421 cm⁻¹ in (CPP-CS-Qu (US) (15:15:1)) and 1422 cm⁻¹ to 1421 cm⁻¹ after ultrasonic treatment, respectively, and 1423 cm⁻¹ in (CPP-CS-Qu (US) (5:5:1)). It could also be seen from the figure that the peak intensity of the amide II band and the peak intensity in the region of 1420-1423 cm⁻¹ increased significantly after ultrasonic treatment. These results indicated that ultrasonic waves formed stronger and more stable hydrogen bonds and electrostatic interactions between casein phosphopeptide, quercetin and chitosan.

(3) X-Ray Diffraction Analysis

Experimental conditions: The working parameters were set as copper target Kα radiation (λ=0.15418 nm), measuring tube pressure 40 kV, tube flow 40 mA, scanning range 5°-80° (2θ), scanning speed 5°/min. The scan mode was continuous.

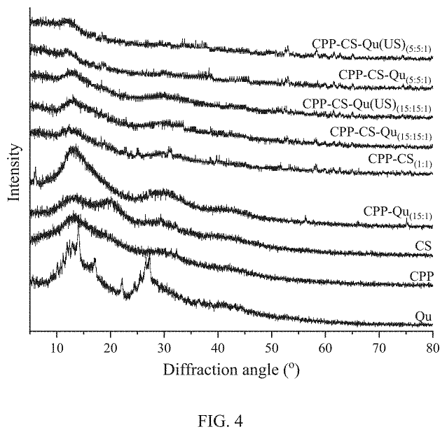

The crystal diffractogram of the sample in FIG. 4 was used to analyze the crystalline or amorphous nature of the substance. Casein phosphopeptide and chitosan exhibited broad humps at diffraction angles (2θ) of 13.1°, 30.5° and 12.8°, 20.2°, and 29.4°, respectively, indicating their amorphous nature. Pure quercetin powder had multiple sharp crystallization peaks between 8.0° and 32.0° in 2θ, indicating that it had a high degree of crystallinity. However, the crystalline characteristic of quercetin nanoparticles CPP-Qu and CPP-CS-Qu almost disappeared in the diffractograms. This result demonstrated that quercetin had been successfully encapsulated within amorphous nanoparticles. The interaction between quercetin, casein phosphopeptides and chitosan molecules may have disrupted the crystal structure of quercetin. The solubility of chemicals in water was affected by its crystalline state. Due to its extremely crystalline structure, the solubility of quercetin in water was restricted. However, when it was converted into an amorphous state in the polypeptide-polysaccharide complex, the solubility was considerably enhanced.

Compared to casein phosphopeptide and chitosan individually, the peak of CPP-CS (1:1) nanoparticles disappeared at 20.2° of 2θ, and the peak intensity around 13° and 30° was significantly diminished. This also revealed a non-covalent interaction between casein phosphopeptide and chitosan, corroborating the infrared spectroscopy results. In addition, FIG. 4 demonstrated that the addition of quercetin had a certain effect on the structure of CPP-CS-Qu nanoparticles. As the mass ratio of quercetin to casein phosphopeptide increased from 1:15 to 1:5, the peak at 13.0° shifted to 11.7° and the peak intensity reduced dramatically. Compared to the control, the sonicated CPP-CS-Qu (US) (5:5:1) nanoparticles did not shift the peak characteristic despite a modest increase in the intensity of the diffraction peak at 11.7°. These results indicated that the crystal structure of CPP-CS-Qu nanoparticles was unaffected by sonication.

### Experimental Example 2: Determination and Investigation of Physicochemical Characteristics of Composite Nanoparticles Loaded with Quercetin

(1) Determination of the Solubility of Quercetin in Water

Determination method: The quercetin concentration in the quercetin-loaded composite nanoparticle dispersion liquid was determined in the same manner as described in the examples, and calculation was done using the standard curve and dilution ratio. And the solubility of quercetin in water was calculated according to the following formula:

Solubility (μg/mL)=detected quercetin concentration×dilution factor

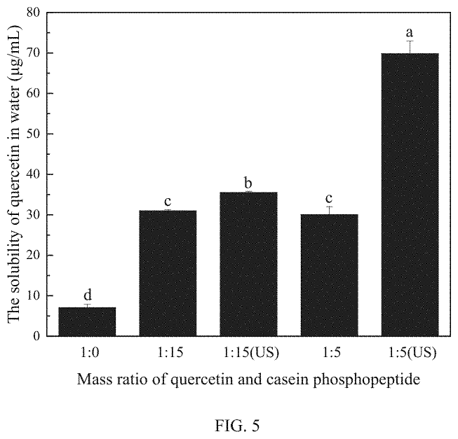

FIG. 5 depicts the influence of different preparation procedures on the solubility of quercetin in water. When the mass ratio of quercetin to casein phosphopeptide was 1:0, the solubility of encapsulated quercetin in water was 7.05 μg/mL. After the addition of casein phosphopeptide and chitosan, the water solubility of quercetin was significantly (P<0.05) improved. In contrast, when the mass ratio of quercetin to casein phosphopeptide was 1:15, the solubility of quercetin in water reached 30.98 μg/mL, which increased to 35.50 μg/mL following sonication. The solubility of quercetin in water was only 30.50 μg/mL when the mass ratio of quercetin to casein phosphopeptide was adjusted to 1:5. However, this value was significantly increased to 69.81 ng/mL after sonication, which was nearly 2.29 times higher than without ultrasound.

(2) Stability of Quercetin in Quercetin Composite Nanoparticles (Considering the Mass Ratio of Quercetin and Casein Phosphopeptide which is 1:15 as an Example)

\textcircled{1} Effect of Temperature on the Stability of Quercetin in Quercetin-Loaded Composite Nanoparticles

Experimental method: The newly prepared free quercetin and quercetin-loaded composite nanoparticle dispersions were placed into glass-sealed bottles and heated in a water bath at 60, 75 and 90° C. for 30 min, respectively. It was then immediately cooled to room temperature at 25° C., and the amount of quercetin remaining in the sample was determined. The retention rate (%) of quercetin was calculated according to the following formula:

$$Quercetin retention rate ( \% ) = \frac{\begin{matrix}Quercetin concentration in \\ the sample after heating\end{matrix}}{\begin{matrix}Quercetin concentration in \\ the sample before heating\end{matrix}} \times 100 \%$$

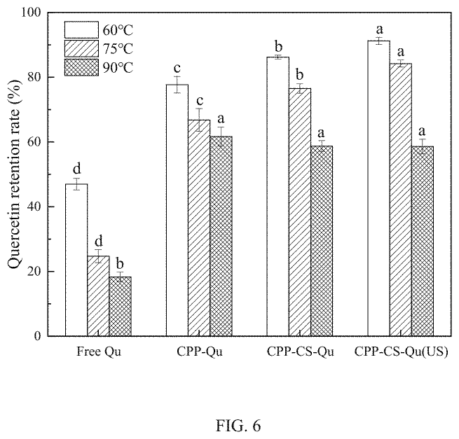

In FIG. 6, it can be clearly seen that the quercetin free in water exhibited poor thermal stability after heat treatment at 60, 75 and 90° C. Nevertheless, when quercetin was encapsulated to produce composite nanoparticles, quercetin retention rates improved significantly (P<0.05). And the thermal stability of encapsulating quercetin with casein phosphopeptide and chitosan was better than that of single casein phosphopeptide. This might be because the complexation of casein phosphopeptide and chitosan results in a denser structure of composite nanoparticles, which may provide better protection for quercetin. The quercetin in the CPP-CS-Qu(US) nanoparticle dispersion prepared by ultrasonic treatment revealed strong thermal stability. When the heat treatment temperature was 60° C. and 75° C., the retention rate of quercetin was significantly increased by 5.03% and 7.71%, respectively, compared to CPP-CS-Qu nanoparticles. This suggested that sonication might dive the development of new structures with higher thermal stability, making it more difficult to disrupt macromolecular structures.

\textcircled{2} Effect of Light on Quercetin Stability in Quercetin-Loaded Composite Nanoparticles

Experimental method: The freshly prepared free quercetin and quercetin-loaded composite nanoparticle dispersions were taken into transparent glass bottles in equal volume. It was then placed in a light cabinet of 0.24 m³ and exposed to light with a wavelength of 253.7 nm, which was generated by a UV lamp with a power of 20 W. After exposure to light for 0, 30, 60, 90, 150, and 210 min, the stability of quercetin in different samples was studied, the residual quercetin content in the samples was determined, and the retention rate of quercetin (%) was calculated according to the following formula:

$$Quercetin retention rate ( \% ) = \frac{\begin{matrix}Quercetin concentration \\ in the sample \\ after illumination\end{matrix}}{\begin{matrix}Quercetin concentration \\ in the sample \\ before illumination\end{matrix}} \times 100 \%$$

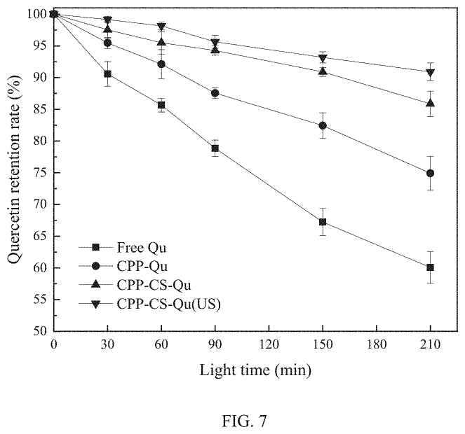

The rate of degradation of dissociated quercetin in water increased with the duration of illumination, as seen in FIG. 7. The retention rate was only 60.08% after 210 min of ultraviolet light irradiation.

In contrast, the encapsulated quercetin showed higher stability to UV radiation. This might be due to the fact that functional groups such as aromatic side chain groups and double bonds of proteins/polypeptides could absorb UV light, thereby boosting the protection of quercetin. The stability of quercetin in CPP-CS-Qu (US) nanoparticles was significantly better than that of CPP-CS-Qu nanoparticles, which might be related to the higher encapsulation efficiency of quercetin-loaded composite nanoparticles prepared by ultrasonic treatment. More quercetin molecules were nano-encapsulated, resulting in less quercetin free in water, hence enhancing the protection for quercetin.

(3) Antioxidant Activity of Quercetin Composite Nanoparticles (Taking the Mass Ratio of Quercetin and Casein Phosphopeptide as 1:15 as an Example)

\textcircled{1} DPPH free radical scavenging method:

A 100 μM solution of DPPH (as-is) was prepared in dark using absolute ethanol. A volume of 2 mL DPPH solution was taken out and mixed well with 2 mL of sample solution, and allowed to react in dark at room temperature for 30 min. The absorbance at 517 nm of the mixture was measured using a UV-visible spectrophotometer. Distilled water and absolute ethanol were served as blank controls instead of samples. The formula for calculating the DPPH free radical scavenging rate of the sample was as follows:

$$DPPH free radical scavenging rate ( \% ) = ( 1 - \frac{A 2 - A 1}{A 0} ) \times 100 \%$$

Wherein, A₀ represents blank; A₁ is the sample with ethanol; A₂ is the sample with DPPH solution.

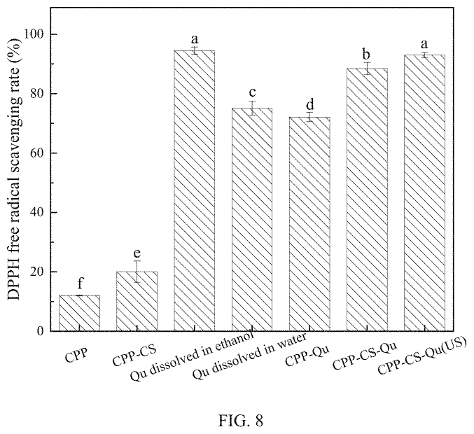

It could be seen from FIG. 8 that the DPPH free radical scavenging rate of quercetin free in ethanol was as high as 94.52%. The strong free radical scavenging activity of quercetin was mainly attributed to the phenolic hydroxyl groups. The DPPH radical scavenging ability of CPP-CS-Qu (US) nanoparticles prepared by sonication was approximately equal to that of Qu-ethanol solution. Compared to the CPP-CS-Qu nanoparticles prepared without sonication, DPPH radical scavenging activity was significantly improved by 4.55%. It might be due to the physical shear force produced by the acoustic cavitation effect generated by ultrasound, which caused the composite nanoparticles have the advantages of small particle size, high encapsulation efficiency, and improved quercetin dispersibility in water. On the other hand, ultrasound may alter the spatial structure of casein phosphopeptide and chitosan, thereby enhancing the antioxidant capacity of the complex. The DPPH free radical scavenging ability of single casein phosphopeptide and CPP-CS nanoparticles was weak, indicating that the antioxidant activity of the complex was mainly attributable to the antioxidant activity of quercetin molecules. After nano-encapsulation, the difficulty of poor water solubility of quercetin was significantly alleviated, and the composite nanoparticles containing quercetin exhibited excellent water dispersibility. This provided a better microenvironment for quercetin, allowing more phenolic hydroxyl groups to be exposed, enabling more free radical scavenging.

\textcircled{2} ABTS free radical scavenging method: 7.4 mM ABTS solution and 2.6 mM potassium persulfate solutions were prepared with deionized water. The two were mixed in equal volumes and reacted at room temperature in the dark for 12-16 h to form an ABTS free radical stock solution. Prior to use, the aforementioned mixture was diluted with 10 mM, pH 6.0 phosphate buffered saline (PBS) at room temperature to obtain ABTS free radical working solution in 0.70±0.02 absorbance at 734 nm. A volume of 100 μL of sample was added to 4 mL of ABTS free radical working solution, mixed thoroughly and allowed to react in the dark for 6 min. The absorbance of test sample was then measured at 734 nm. Deionized water and absolute ethanol were used as blank controls instead of samples. The formula to calculate the free radical scavenging rate of ABTS was as follows:

$$ABTS free radical scavenging rate ( \% ) = ( 1 - \frac{AS}{AC} ) \times 100 \%$$

In the formula, AC is the absorbance ABTS working solution with ethanol; AS is ABTS working solution with sample.

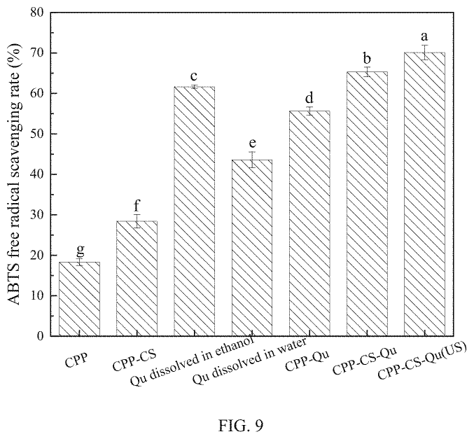

As shown in FIG. 9, the antioxidant capacity of the casein phosphopeptide single without quercetin and CPP-CS nanoparticles was relatively weak. After the casein phosphopeptide and chitosan nano-encapsulation, the ABTS free radical scavenging ability of the quercetin-loaded composite nanoparticles was significantly improved by 12.03%-26.52% (P<0.05) compared to the free quercetin in water. This was because encapsulation increased dispersibility and solubility of quercetin molecules in water. Among them, the CPP-CS-Qu (US) nanoparticles prepared by ultrasonic treatment exhibited the highest ABTS radical scavenging rate of 70.11%. In conclusion, the encapsulation of quercetin did not impede its own scavenging activity significantly.

(4) The Release Properties of Quercetin During In Vitro Gastrointestinal Digestion Simulation (Considering the Mass Ratio of Quercetin and Casein Phosphopeptide as 1:15 as an Example)

Experimental method: The pH of 40 mL of freshly prepared free quercetin and quercetin composite nanoparticles dispersion was adjusted to 2.0 with 1 M HCl. After 10 min of preheating in a shaker (37° C., 100 rpm/min), 26.7 mg of pepsin was added and thoroughly mixed for 1 h to initiate simulated gastric digestion. After 1 h of digestion at 37° C., the pH of the pepsin digest was adjusted to 7.4 using 5 M NaOH. 200 mg of bile salts were added and mixed well in a shaker for 10 min. Then 13.6 mg of trypsin was added to start simulated intestinal digestion for 2 h. During the simulated gastrointestinal digestion process, 3 mL of digestion samples were collected at different digestion time (0, 30, 60, 90, 120, 150 and 180 min), and the enzyme was immediately inactivated with liquid nitrogen to terminate the reaction. The content of quercetin was determined, and the release rate (%) of quercetin was calculated according to the following formula:

$$Quercetin release rate ( \% ) = [ 1 - \frac{\begin{matrix}Quercetin content in \\ digestive juice\end{matrix}}{\begin{matrix}Total quercetin content \\ in samples before \\ digestion\end{matrix}} ] \times 100 \%$$

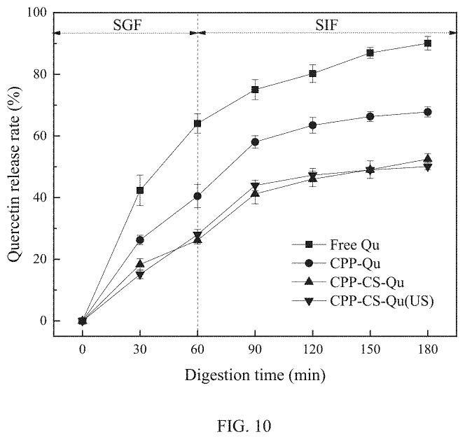

FIG. 10 showed the release characteristics of quercetin in different systems that imitate in vitro digestion gastrointestinal tract. The stability of free quercetin in the digestive juice of the gastrointestinal tract was quite poor, as depicted in the figure. Especially when simulating gastric digestion release rate was significantly higher. In the first 30 min, it was 42.36% which reached to 64.06% at 60 min, indicating sudden release in the stomach.

After 60 minutes of digestion in the stomach, the release rate of encapsulated quercetin nanoparticles of different systems decreased dramatically. This indicated that casein phosphopeptide and chitosan were effective encapsulation carriers, resulting in a considerable delay of quercetin release of quercetin in the stomach. However, the encapsulated quercetin likewise exhibited a burst release phenomena during the first 30 min of simulated intestinal digestion, followed by a slowing of the release rate. This might be attributed to a change in system pH from 2.0 to 7.4 that occurs during the transition from the stomach to the intestinal environment. The composite nanoparticles loaded with quercetin underwent a series of structural changes, resulting in the release of a part of the quercetin in the process. Subsequent intestinal digestion, 90.09% of free quercetin was released in the digestive juice; however, the release rate of encapsulated quercetin was significantly lower than that of free quercetin. According to the results, ultrasound had no significant effect on the digestion and release properties of quercetin in polypeptide-polysaccharide composite nanoparticles. The sonicated nanoparticles might also meet the goal of quercetin being retained in the stomach and being released gradually and effectively in the intestine, which would be beneficial to boost quercetin bioavailability.

What is claimed is:

1. An ultrasonic preparation method of protein-derived peptide-polysaccharide nanoparticles loaded with bioactive components, comprising the following steps:
(1) dissolving a casein phosphopeptide in a distilled water to obtain a casein phosphopeptide solution, wherein a pH of the casein phosphopeptide solution is adjusted to 11;
(2) dissolving a chitosan in a 1% glacial acetic acid solution to obtain a chitosan solution, and stirring the chitosan solution magnetically to dissolve the chitosan completely;
(3) dissolving a quercetin into an absolute ethanol to obtain a quercetin stock solution;
(4) according to different mass ratios of the quercetin to the casein phosphopeptide, the casein phosphopeptide solution in the step (1) is slowly added to the quercetin stock solution in the step (3) under a constant stirring to obtain a mixture;
(5) according to different mass ratios of the casein phosphopeptide to the chitosan, an equal volume of the chitosan solution of the step (2) is added dropwise to the mixture of the step (4) under a constant stirring at a room temperature to obtain a mixed solution; wherein a pH of the mixed solution is adjusted to 6; and
(6) performing an ultrasonic treatment on the mixed solution in the step (5), wherein once the ultrasonic treatment is completed, a quercetin-loaded casein phosphopeptide-chitosan nanoparticle dispersion is collected; finally, after a freeze-drying, a quercetin-loaded casein phosphopeptide-chitosan nanoparticle is obtained.

2. The ultrasonic preparation method of the protein-derived peptide-polysaccharide nanoparticles loaded with the bioactive components according to claim 1, wherein a concentration of the casein phosphopeptide in the step (1) is 1.0 mg/ml-3.0 mg/ml.

3. The ultrasonic preparation method of the protein-derived peptide-polysaccharide nanoparticles loaded with the bioactive components according to claim 1, wherein a mass ratio of the quercetin to the casein phosphopeptide specified in the step (4) is between 1: 5-1:20.

4. The ultrasonic preparation method of the protein-derived peptide-polysaccharide nanoparticles loaded with the bioactive components according to claim 1, wherein a mass ratio of the casein phosphopeptide to the chitosan in the step (5) is (1-3):(1-3).

5. The ultrasonic preparation method of the protein-derived peptide-polysaccharide nanoparticles loaded with the bioactive components according to claim 1, wherein specific parameters of the ultrasonic treatment in the step (6) are ultrasonic frequencies of 20 kHz, 35 kHz, 50 kHz, 20/35 kHz, 20/50 kHz, 35/50 kHz, 20/35/50 kHz, an ultrasonic power of 60 W-300 W, an ultrasonic time of 5 min-30 min, an ultrasonic interval ratio of 30 s/5 s.

6. The ultrasonic preparation method of the protein-derived peptide-polysaccharide nanoparticles loaded with the bioactive components according to claim 2, wherein the concentration of the casein phosphopeptide in the step (1) is 1.5 mg/ml.

7. The ultrasonic preparation method of the protein-derived peptide-polysaccharide nanoparticles loaded with the bioactive components according to claim 3, wherein the mass ratio of the quercetin and the casein phosphopeptide described in the step (4) is 1:15.

8. The ultrasonic preparation method of the protein-derived peptide-polysaccharide nanoparticles loaded with the bioactive components according to claim 4, wherein the mass ratio of the casein phosphopeptide to the chitosan stated in the step (5) is 1:1.

9. The ultrasonic preparation method of the protein-derived peptide-polysaccharide nanoparticles loaded with the bioactive components according to claim 5, wherein the specific parameters of the ultrasonic treatment in the step (6) are an ultrasonic frequency of 35/50 kHz; the ultrasonic power of 240 W; and the ultrasonic time of 15 min.
